(19) United States
(12) Patent Application Publication
(10) Pub. No.: US 2016/0223557 A1
(43) Pub. Date: Aug. 4, 2016

(54) METHOD OF ISOLATING CIRCULATING TUMOR CELLS

(71) Applicants: George V. Thomas, Lake Oswego, OR (US); Jennifer Podolak, Portland, OR (US)

(72) Inventors: George V. Thomas, Lake Oswego, OR (US); Jennifer Podolak, Portland, OR (US)

(73) Assignee: OREGON HEALTH & SCIENCE UNIVERSITY, PORTLAND, OR (US)

(21) Appl. No.: 15/014,388

(22) Filed: Feb. 3, 2016

Related U.S. Application Data
(60) Provisional application No. 62/112,034, filed on Feb. 4, 2015.

(51) Int. Cl. G01N 33/574; G01N 1/34

(52) U.S. Cl. CPC G01N 33/57492; G01N 1/34; G01N 2333/70596

(57) ABSTRACT

A method of purifying circulating tumor cells is disclosed. The method involves purifying a first buffy coat layer from a blood sample on a first density gradient. The blood sample comprises a circulating tumor cell. The first buffy coat layer is reconstituted in whole blood to yield an enriched sample. The volume of whole blood used to reconstitute the first buffy coat layer is less than 20% of the volume of the blood sample. Circulating tumor cell enrichment reagent is added to the enriched sample at a volume of less than 20% of the total volume of the enriched sample. The enriched sample is placed on a second density gradient and a second buffy coat layer is collected. The second buffy coat layer comprises the circulating tumor cell.

### ACKNOWLEDGEMENT OF GOVERNMENT SUPPORT

This invention was made with the support of the United States government under the terms of an ARRA grant. The United States government has certain rights to this invention.

### FIELD

Generally, the field is the isolation of cell populations. More specifically, the field is the isolation of circulating tumor cells from blood.

### SUMMARY

Circulating tumor cells have the potential to provide early, noninvasive detection of tumors. However, reagents and systems used in isolating circulating tumor cells from blood are expensive and complex. Many of these systems are beyond the reach of research laboratories. Disclosed herein is a method of isolating circulating tumor cells using as little as 1/30 of the amount of circulating tumor cell enrichment reagent without detrimental effects on the number of circulating tumor cells collected.

The method involves purifying a first buffy coat layer from a blood sample on a first density gradient. The blood sample comprises a circulating tumor cell. The first buffy coat layer is reconstituted in whole blood to yield an enriched sample. The volume of whole blood used to reconstitute the first buffy coat layer is less than 20% of the volume of the blood sample. Circulating tumor cell enrichment reagent is added to the enriched sample at a volume of less than 20% of the total volume of the enriched sample. The enriched sample is placed on a second density gradient and a second buffy coat layer is collected. The second buffy coat layer comprises the circulating tumor cell.

Also described are circulating tumor cells isolated by the disclosed method.

### BRIEF DESCRIPTION OF THE SEVERAL VIEWS OF THE DRAWINGS

The drawings herein are better understood when presented in color, which is not available in patent application publications. However, Applicants consider the color drawings to be part of the original disclosure and reserve the right to present color versions of the drawings herein in later proceedings.

FIG. 1 is a set of images illustrating the multicolor immunofluorescence imaging parameters establishing CTC detection using LNCaP cells spiked into control blood specimens, that were captured using the disclosed methods and cytospun onto charged slides, and subsequently stained with antibodies against pan-cytokeratin, CK and/or PSA (CTC marker), CD45 (leukocyte marker) and Dapi (nuclear marker). PSA+/CK+/Dapi+/CD45− cells are scored as CTCs

FIG. 2 is a set of images using the parameters illustrated in FIG. 1 to detect patient derived CTCs in blood from a prostate cancer patient. In particular, FIG. 2 shows Dapi+/PSA+/CK+/CD45− CTCs captured from a CRPC patient using the disclosed method.

FIG. 3 is a set of images demonstrating parameters for single cell AR signaling established as in FIG. 1 by staining captured CTCs with a dual FISH-IF protocol to document AR amplification and AR expression, as seen in CTCs captured from another CRPC patient using the disclosed method.

### DETAILED DESCRIPTION

Disclosed herein is a method of isolating circulating tumor cells in a blood sample. The method involves purifying a first buffy coat layer from a blood sample on a density gradient. The blood sample has a first volume. The first buffy coat layer is reconstituted in a second volume of whole blood. This yields an enriched sample. The volume of enriched sample is no more than 20% of the volume of the original blood sample. The circulating tumor cell enrichment reagent is added to the enriched sample at a volume less than 20% that of the enriched sample. The circulating tumor cells are then purified by purifying a second buffy coat layer from the enriched sample on a second density gradient.

The blood sample can be any sample of venous or arterial blood from a subject that contains or could contain a circulating tumor cell. The volume of the blood sample can be any appropriate volume. For human subjects, an appropriate volume can be more than 2 ml, more than 5 ml, more than 10 ml, more than 20 ml, more than 25 ml, more than 50 ml, more than 100 ml, more than 200 ml, or more than 400 ml. Subjects also include veterinary subjects as well as laboratory animals. Appropriate volumes of blood samples can be adjusted based on the size of the subject. The blood sample can be collected by any appropriate method, such as collection in a tube. Appropriate additives can be added to the blood sample such as anti-coagulants, preservatives, buffers (such as PBS) or other additives. Such additives necessarily increase the volume of the blood sample, but a blood sample with additives is still referred to as a blood sample herein.

A buffy coat layer results from the centrifugation of blood on a density gradient. For the purposes of this application, a density gradient is a composition that promotes the separation of blood components such as plasma, erythrocytes, and buffy coat during centrifugation. Density gradients can comprise polymeric compounds such as polyvinylpyrrolidine-coated colloidal silica (Percoll®); 3,5-diacetamido-2,4,6-triiodobenzoic acid (Ficoll®); sugars such as sucrose and dextran; proteins such as BSA; iodinated low molecular weight compounds (metrizamide); or heavy salts such as cesium chloride. Density gradients are adjusted to have proper density, osmolality, and pH. One of skill in the art in light of this disclosure will be able to select and/or modify a density gradient for the desired purpose herein. In general, when whole blood is placed on a density gradient, the buffy coat separates in the middle of the gradient, above the erythrocyte layer and below the plasma layer. Circulating tumor cells separate with the buffy coat.

The second volume of whole blood in which the first buffy coat layer is reconstituted is no more than 20% of the original volume of the blood sample. It can also be no more than 15% of the original volume, no more than 10%, no more than 5%, No more than 3.5%, no more than 3.3%, no more than 3%, no more than 2%, no more than 1%, or less than 1% of the original volume of the blood sample. For example, if the original volume of the blood sample is 30 ml, then the volume of whole blood in which the first buffy coat layer is reconstituted is no more than 6 ml. If the original volume of the blood sample is 15 ml, then the volume of blood in which the first buffy coat layer is reconstituted is no more than 3 ml. In a particular example, the original volume of the blood sample is 30 ml and the volume of whole blood in which the first buffy coat layer is reconstituted is 1 ml. The whole blood from which the first buffy coat layer is reconstituted can be from any source. In some examples, the second volume of whole blood is reserved from the original sample. In other examples, the second volume of whole blood is from a healthy human subject or from a veterinary subject. Additives such as those described above can be added to the enriched sample.

The circulating tumor cell enrichment reagent can be any reagent that enriches for circulating tumor cells. The reagents can positively select circulating tumor cells by binding them directly or negatively select circulating tumor cells by binding cells known not to be circulating tumor cells. Such reagents can include antibodies such as anti-CD36 antibodies, anti-EpCAM antibodies, anti-CD45 antibodies, and others. Such reagents can also comprise elements that aid in the separation of the tumor cells. Examples include fluorescent labels, which allow selection via flow cytometry or other methods; magnetic beads, which allow positive selection of circulating tumor cells using a magnet; or structures that promote the formation of immunorosettes, which are tetrameric antibody structures that allow positive or negative selection of cells through the formation of structures that include red blood cells, thereby increasing the size of the unwanted cell and allowing further separation through any of a number of methods (Thomas TE et al, U.S. Pat. No. 7,135,335 (2006); and Stemcell Technologies Document Number 28583, version 1.2.0, available at

http://www.stemcell.com/en/Products/All-Products/Tumor-Cell-Enrichment-Cocktail.aspx (2014); both of which are incorporated by reference herein.)

The enriched sample is placed on a second density gradient which can be either the same density gradient or a different density gradient. For example, both density gradients can comprise Ficoll-Paque®. After purification on the second density gradient, a volume of an appropriate red blood cell lysis buffer can be added to the circulating tumor cells purified from the second density gradient. Numerous red cell lysis buffers are known in the art.

### EXAMPLES

The following examples are illustrative of the disclosed methods. In light of this disclosure, those of skill in the art will recognize that variations of these examples and other examples of the disclosed methods would be possible without undue experimentation.

### Example 1

### Example CTC Isolation Procedure

A blood sample is collected in BD Vacutainer® K2 EDTA collection tubes. A 15 ml volume of Ficoll-Paque™ Plus is added to a Leucosep™ tube to form the first density gradient. This is then spun in a centrifuge at 1000×g for 1 minute. A 1 ml volume of blood sample is then reserved to later reconstitute the first buffy coat layer. The remaining blood sample is added to a conical tube and PBS added to bring the total volume to 30 ml. The blood sample+PBS is then added to the first density gradient. The blood sample on the first density gradient is centrifuged at 1600×g for 20 minutes at room temperature with the centrifuge brake disengaged.

The buffy coat is collected from the first density gradient and then spun at 400×g for four minutes to pellet the cells. The 1 ml of reserved blood is added to the cells from the first density gradient to create the enriched sample, pipetting to break up the pellet. A 50 μl volume of RossetteSep® Human Circulating Epithelial Tumor Cell Enrichment Cocktail (Stemcell

Technologies Cat #15127) is added to the enriched sample. This is incubated at room temperature for at least 20 minutes. Then the volume of the sample is brought to a total of 6 ml by the addition of PBS and this is then added to the second density gradient (4 ml Ficoll-Paque® in a 15 ml conical tube.) The enriched sample on the second density gradient is then centrifuged at 1200×g for 20 minutes at room temperature.

The resulting CTC layer is removed from the second density gradient and transferred to a 15 ml conical tube. PBS is added to bring the volume to 9 ml and 1 ml of a 10× red blood cell lysis buffer is added. The tubes are then agitated for 15 minutes at room temperature until final pelleting by a centrifugation at 400×g for four minutes.

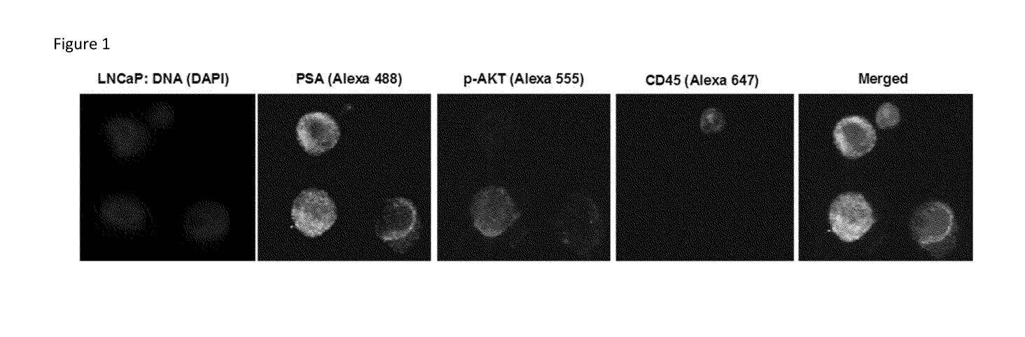

At this point, the cells can be labeled, fixed in formaldehyde, and/or stained by any of a number of techniques known in the art. FIG. 1 is a proof of principle study using LNCaP cells spiked into blood and isolated with the disclosed method. FIG. 1 shows the captured LNCaP cells with nuceli stained with DAPI (far left, blue); PSA (second from left, green) p-AKT Ser473 (middle, red) and CD45 (second from right, pink). The rightmost image is the merged image of LNCAP cells with a DAPI+/PSA+/pAKT+/CD45− phenotype.

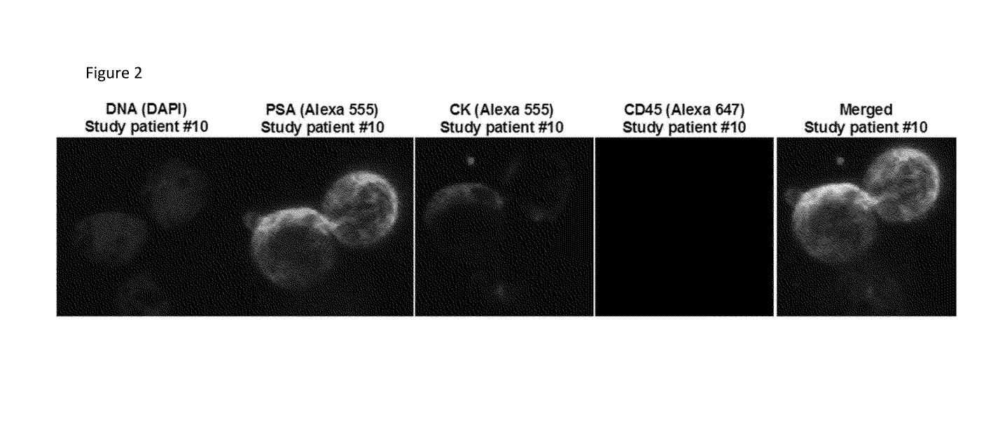

FIG. 2 is a demonstration that CTC's can be captured using the disclosed method from a prostate cancer patient. CTCs defined by the immunophenotype Dapi+/PSA+/CK+/CD45− were isolated from a patient. To achieve multiparameter single-cell analysis of AR and P13K activity, we developed an automated fluorescence microscopy scanning platform using the 3DH Panoramic scanner and automatically quantified immunofluorescence using the inFORM hyperspectral image analysis platform.

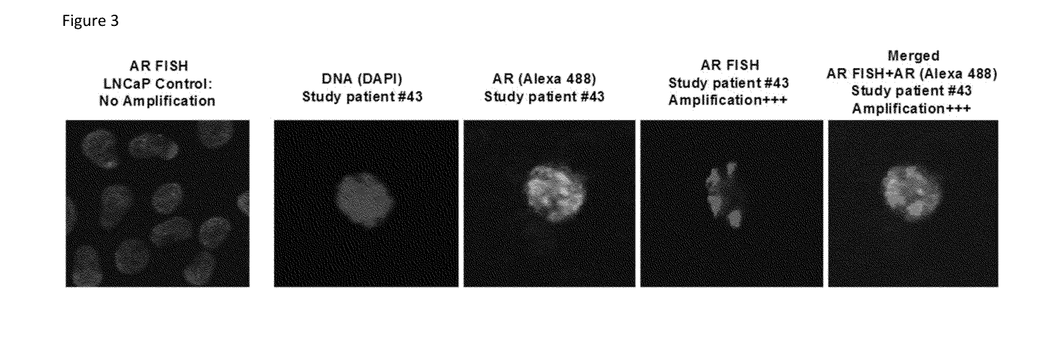

FIG. 3 is a showing of AR amplification in the CTC's captured using the disclosed method from a prostate cancer patient sample. Nuclei (blue) of a captured CTC from study patient #43 co-expresses AR immunofluorescence (green) and FISH with AR gene locus (red). The LNCaP control on far left shows normal AR signal, i.e. 2 distinct red probes.

1. A method of isolating a circulating tumor cell, the method comprising:
purifying a first buffy coat layer from a blood sample on a first density gradient, wherein the blood sample comprises a first volume and wherein the blood sample comprises a circulating tumor cell;
reconstituting the first buffy coat layer in a second volume of whole blood to yield an enriched sample, wherein the second volume is less than 20% of the first volume;
adding a third volume of circulating tumor cell enrichment reagent to the enriched sample wherein the third volume is less than 20% of the second volume;
purifying a second buffy coat layer from the enriched sample on a second density gradient, thereby isolating the circulating tumor cell.

2. The method of claim 1 wherein the purifying the second buffy coat layer from the enriched sample occurs after the adding the CTC enrichment reagent to the enriched sample.

3. The method of claim 1 wherein the second volume is less than 5% of the first volume and the third volume is less than 5% of the second volume.

4. The method of claim 3 comprising adding a volume of PBS to the blood sample.

5. The method of claim 4 wherein the first volume is 30 ml, wherein the second volume is 1 ml and wherein the third volume is 0.05 ml.

6. The method of claim 1 wherein the second volume of whole blood is set aside from the blood sample.

7. The method of claim 1 wherein the first density gradient and the second density gradient comprise Ficoll-Paque™.

8. The method of claim 1 further comprising adding a fourth volume of red blood cell lysis buffer to the enriched sample.

9. The method of claim 1 wherein the circulating tumor cell enrichment reagent comprises an anti-CD36 antibody.

10. The method of claim 9 wherein the circulating tumor cell enrichment reagent comprises RosetteSep™ CTC Enrichment Cocktail Containing Anti-CD36.

11. Circulating tumor cells isolated by the method of claim 1.
